(19) United States
(12) Patent Application Publication
(10) Pub. No.: US 2025/0340617 A1
(43) Pub. Date: Nov. 6, 2025

(54) COLLAGEN PROCESSING STRUCTURE, COLLAGEN PROCESSING SYSTEM, AND METHOD FOR PROCESSING COLLAGEN

(71) Applicant: HUIZHOU FORYOU MEDICAL DEVICES CO., LTD., HUIZHOU (CN)

(72) Inventors: TIANFA DENG, HUIZHOU (CN); YINGYING ZHU, HUIZHOU (CN); HENGMING ZHANG, HUIZHOU (CN)

(21) Appl. No.: 19/266,140

(22) Filed: Jul. 10, 2025

Related U.S. Application Data
(63) Continuation of application No. PCT/CN2024/105547, filed on Jul. 15, 2024.

(30) Foreign Application Priority Data
Apr. 26, 2024 (CN) 202410517570.1

(51) Int. Cl. C07K 14/78; B01D 61/14; B01D 61/18; B01D 63/16; B01D 69/02; B01D 71/02

(52) U.S. Cl. CPC C07K 14/78; B01D 61/145; B01D 61/18; B01D 63/16; B01D 69/02; B01D 71/02; B01D 2313/243; B01D 2313/26; B01D 2313/46; B01D 2325/02833

(57) ABSTRACT

A collagen processing structure, a collagen processing system, and a method for processing collagen. The collagen processing structure includes: a processing chamber, defining a discharge outlet; and a centrifugal processing mechanism, including a centrifugal driving member and a centrifugal ultrafiltration member. The centrifugal ultrafiltration member is connected to the centrifugal driving member. The centrifugal driving member is configured to drive the centrifugal ultrafiltration member to rotate. The centrifugal ultrafiltration member is disposed in the processing chamber. An ultrafiltration membrane layer is arranged on each of two opposite sides of the centrifugal ultrafiltration member. A filtration chamber is defined in the centrifugal ultrafiltration member. A waste liquid hole is defined on the centrifugal ultrafiltration member, and is in communication with the filtration chamber and the discharge outlet.

### CROSS-REFERENCE TO RELATED APPLICATIONS

The present application is a continuation-application of International (PCT) Patent Application No. PCT/CN2024/105547, filed Jul. 15, 2024, which claims foreign priority of Chinese Patent Application No. 202410517570.1, filed on Apr. 26, 2024, and both of which are hereby incorporated by reference in their entireties.

### TECHNICAL FIELD

The present disclosure relates to the field of biomaterials, and in particularly to a collagen processing structure, a collagen processing system, and a method for processing collagen.

### BACKGROUND

Collagen is a macromolecular protein having three chains, with characteristics such as a high viscosity, a poor water solubility, and easy gel formation. Especially during purification and concentration processes, a viscosity of the collagen may be increased rapidly, which leads to difficulty in implementing and operating to obtain high-concentration collagen (e.g., exceeding 20 mg/ml). Currently, in a collagen production, methods such as salting-out, dialysis, ultrafiltration, combined methods, etc., are commonly applied to purify and concentrate the collagen. However, the above methods for purifying and concentrating the high-concentration collagen have disadvantages such as low purity, low efficiency, susceptibility to contamination, complex techniques, etc.

### SUMMARY OF THE DISCLOSURE

According to a first aspect, some embodiments of the present disclosure provide a collagen processing structure. The collagen processing structure includes: a processing chamber, defining a discharge outlet; and a centrifugal processing mechanism, including a centrifugal driving member and a centrifugal ultrafiltration member, where the centrifugal ultrafiltration member is connected to the centrifugal driving member, and the centrifugal driving member is configured to drive the centrifugal ultrafiltration member to rotate; where the centrifugal ultrafiltration member is disposed in the processing chamber, and an ultrafiltration membrane layer is arranged on each of two opposite sides of the centrifugal ultrafiltration member, a filtration chamber is defined in the centrifugal ultrafiltration member, a waste liquid hole is defined on the centrifugal ultrafiltration member and is in communication with the filtration chamber, and the waste liquid hole is further in communication with the discharge outlet.

According to a second aspect, some embodiments of the present disclosure provide a collagen processing system. The collagen processing system includes the above-mentioned collagen processing structure, a storage tank, a delivering pipeline, a return pipeline, a sewage pipeline, and a water injection pipeline. The delivering pipeline is connected to the storage tank and the collagen processing structure. The return pipeline is connected to the collagen processing structure and the storage tank. The sewage pipeline is connected to the discharge outlet. The water injection pipeline is connected to the storage tank

According to a third aspect, some embodiments of the present disclosure provide a method for processing collagen. The method for processing collagen includes: delivering an initial collagen extraction solution in a storage tank to a collagen processing structure; performing, by a centrifugal processing mechanism of the collagen processing structure, cyclic centrifugal ultrafiltration processing, and purifying the initial collagen extraction solution; and in a case where an electrical conductivity in the storage tank is satisfied with a first preset value, stopping purifying the initial collagen extraction solution. Performing, by the centrifugal processing mechanism of the collagen processing structure, the cyclic centrifugal ultrafiltration processing, and purifying the initial collagen extraction solution, includes: discharging waste liquid generated in each centrifugal ultrafiltration processing, returning collagen liquid extracted in the each centrifugal ultrafiltration processing to the storage tank, and simultaneously supplementing the same amount of water as the waste liquid into the storage tank; delivering mixed liquid to the collagen processing structure again and performing another centrifugal ultrafiltration processing, and repeating until the purifying the initial collagen extraction solution is stopped, where the mixed liquid is formed by remaining initial collagen extraction solution in the storage tank, supplemented water, and the collagen liquid extracted in the centrifugal ultrafiltration processing.

The above description is only an overview of the technical solutions of the present disclosure, and in order to make the technical means of the present disclosure more clear, the present disclosure may be implemented in accordance with the content of the description, and in order to make the above and other objects, features, and advantages of the present disclosure more clear, the following description is given in conjunction with the specific embodiments of the present disclosure.

### BRIEF DESCRIPTION OF THE DRAWINGS

From the following detailed description of the specific embodiments, other advantages and benefits of the present disclosure will be apparent to those skilled in the art. The drawings are only for purposes of illustrating the embodiments and are not to be construed as limiting the present disclosure. Besides, throughout the drawings, same reference numerals refer to the same parts. In the drawings:

FIG. 1 is a schematic structural view of a collagen processing structure according to some embodiments of the present disclosure.

FIG. 2 is a first schematic structural view of a centrifugal ultrafiltration member of the collagen processing structure according to some embodiments of the present disclosure.

FIG. 3 is a second schematic structural view of a centrifugal ultrafiltration member of the collagen processing structure according to some embodiments of the present disclosure.

FIG. 4 is a schematic structural view of a collagen processing system according to some embodiments of the present disclosure.

FIG. 5 is a schematic diagram of a purification process of a method for processing collagen according to some embodiments of the present disclosure.

FIG. 6 is a schematic diagram of a concentration process of the method for processing collagen according to some embodiments of the present disclosure.

Reference numerals in the drawings: 100, collagen processing system; 10, collagen processing structure; 11, processing chamber; 111, discharge outlet; 112, feed inlet; 113, liquid return port; 12, centrifugal processing mechanism; 121, centrifugal driving member; 1211, centrifugal shaft; 1212, hollow channel; 122, centrifugal ultrafiltration member; 1221, ultrafiltration membrane layer; 1222, filtration chamber; 1223, waste liquid hole; 1224, mounting hole; 13, second discharge outlet; 20, storage tank; 21, first discharge outlet; 30, delivering pipeline; 40, return pipeline; 50, sewage pipeline; 60, water injection pipeline; 61, flow meter; 70, pressure-regulating assembly; 72, ultrafiltration pump; 73, flushing pump; 71, compressed air inlet; 80, flushing tank; 90, flushing pipeline.

### DETAILED DESCRIPTION

The embodiments of the technical solutions of the present disclosure may be described in detail below. The following embodiments are only used to illustrate the technical solutions of the present disclosure more clearly, and thus are only examples, and cannot be used to limit the protection scope of the present disclosure.

Unless otherwise defined, all technical and scientific terms used herein have the same meanings as commonly understood by those skilled in the art to which the present disclosure belongs. The terms used herein are only intended to illustrate the purpose of the embodiments of the present disclosure and are not intended to limit the present disclosure. The terms “include/including”, “have/has” and any variations thereof in the description, claims and the above-mentioned attached drawings of the present disclosure are intended to cover non-exclusive inclusion.

In the description of the embodiments of the present disclosure, technical terms such as “first” and “second” are only used to distinguish different objects, and cannot be understood as indicating or implying relative importance, or implicitly indicating the number, specific order or primary-secondary relationship of the indicated technical features. In the description of the embodiments of the present disclosure, unless otherwise clearly and specifically defined, the term “multiple” may be referred to two or more (including two). Similarly, “multiple groups” may be referred to two or more groups (including two groups), and “multiple pieces” may be referred to two or more pieces (including two pieces).

“Embodiment” herein means that a particular feature, structure, or characteristic described with reference to embodiments may be included in at least one embodiment of the present disclosure. The term appearing in various places in the specification are not necessarily as shown in the same embodiment, and are not exclusive or alternative embodiments that are mutually exclusive with other embodiments. Those skilled in the art will understand explicitly and implicitly that the embodiments described herein may be combined with other embodiments.

In the description of the embodiments of the present disclosure, the term “and/or” is simply a description of the association of related objects, indicating that three relationships can exist, e.g., A and/or B, which can mean: A alone, both A and B, and B alone. In addition, the character “/” in this document generally indicates that the before and after associated objects are in an “or” relationship.

In the description of the embodiments of the present disclosure, technical terms such as “middle”, “longitudinal”, “transverse”, “length”, “width”, “thickness”, “upper”, “lower”, “front”, “rear”, “left”, “right”, “vertical”, “horizontal”, “top”, “bottom”, “inner”, “outer”, “clockwise”, “counter-clockwise”, “axial”, “radial”, “peripheral” and others may indicate directions or positions that are based on the orientation or position relationship shown in the drawings, and are only for the convenience of describing the application and simplification of the description, but not indicate or imply that the apparatus or unit referred to must have a specific orientation, be constructed and operated in a specific orientation, therefore cannot be construed as a restriction on this application.

In the description of the embodiments of the present disclosure, unless otherwise clearly and specifically defined, it should be noted that technical terms such as “mount”, “install”, “connect”, and “connection” should be understood in a broad sense. For example, the connection may be a fixed connection, a detachable connection, or an integral connection; or the connection may be a mechanical connection, an electrical connection, or mutual communication; or the connection may be a direct connection, an indirect connection through an intermediary, internal communication between two elements, or interactions between two elements, unless otherwise explicitly defined. Persons of ordinary skill in the art may understand the specific meanings of the foregoing terms in the embodiments of the present disclosure according to specific situations.

Animal-derived collagen may be mainly extracted from animal bones, skin, tendons, membranes, and other tissues. A collagen content of type I collagen is extremely rich, accounting for approximately 80%-90% of the total collagen content. As a main component of an extracellular matrix, the type I collagen has good reparative property, biocompatibility, biodegradability, and other properties. The type I collagen is widely applied in fields such as medicine, food, cosmetology, etc., which may play an important role.

Collagen is generally obtained through the following process: extraction from the tissues via physical, chemical, and biological methods (such as a common acid-enzyme extraction method), and further purification and concentration of the extracted collagen. Currently, in collagen production, a method such as salting-out, dialysis, ultrafiltration, combined methods, etc., may be commonly used to purify and concentrate the collagen.

However, the collagen is a macromolecular protein having three chains, with characteristics such as a high viscosity, a poor water solubility, and easy gel formation. Especially during purification and concentration processes, a viscosity of the collagen may be increased rapidly, which leads to difficulty in implementing and operating to obtain high-concentration collagen (e.g., exceeding 20 mg/ml). The above methods for purifying and concentrating high-concentration collagen have disadvantages such as low purity, low efficiency, susceptibility to contamination, complex techniques, etc.

In view of this, in order to solve the technical problems of the method for purifying and concentrating the collagen in the related art, a collagen processing structure, a collagen processing system, and a method for processing collagen may be provided by some embodiments of the present disclosure, such that it may be possible to improve collagen purity, enhance ultrafiltration efficiency, and employ a simple technology to reach high concentration multiple, i.e., the collagen concentration of up to 20 mg/ml.

For the convenience of description, the following embodiments may take a collagen processing structure in the embodiment of the present disclosure as an example for illustration.

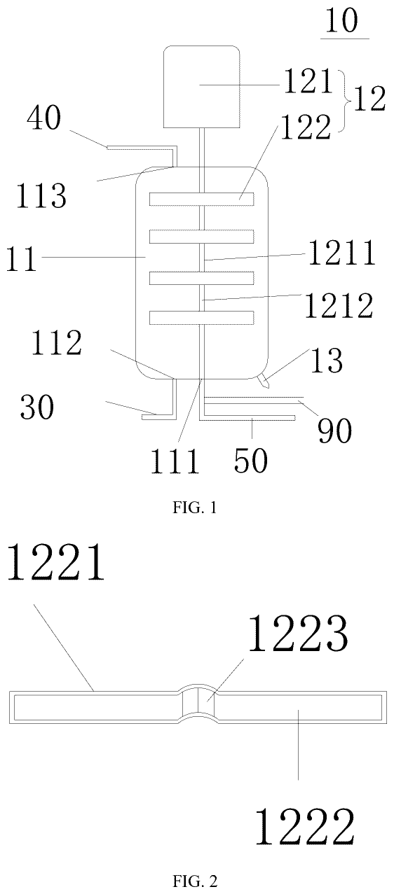

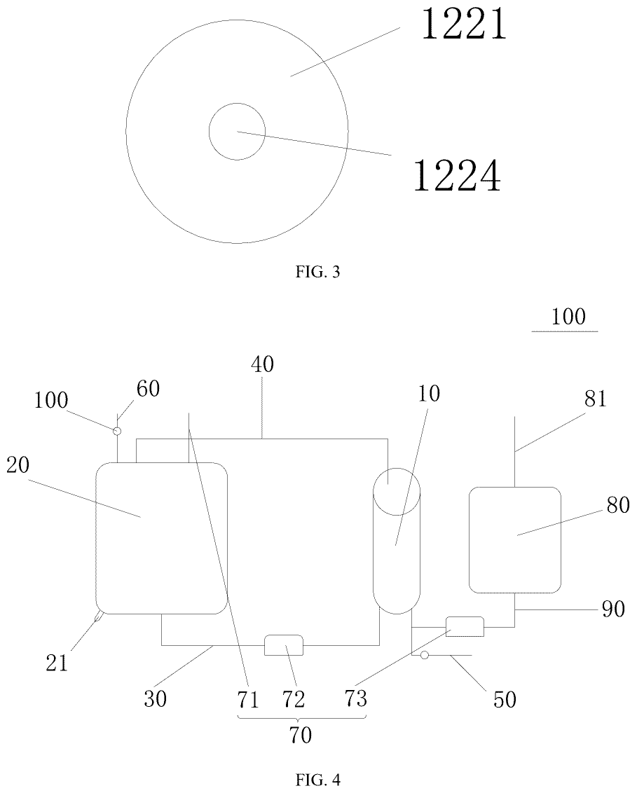

As shown in FIGS. 1 to 3, FIG. 1 is a schematic structural view of a collagen processing structure according to some embodiments of the present disclosure, FIG. 2 is a first schematic structural view of a centrifugal ultrafiltration member of the collagen processing structure according to some embodiments of the present disclosure, and FIG. 3 is a second schematic structural view of a centrifugal ultrafiltration member of the collagen processing structure according to some embodiments of the present disclosure. According to an aspect, a collagen processing structure 10 may be provided by some embodiments of the present disclosure. The collagen processing structure 10 may include a processing chamber 11 and a centrifugal processing mechanism 12. A discharge outlet 111 may be defined on the processing chamber 11. The centrifugal processing mechanism 12 may include a centrifugal driving member 121 and a centrifugal ultrafiltration member 122. The centrifugal ultrafiltration member 122 may be connected to the centrifugal driving member 121, and the centrifugal driving member 121 may be configured to drive the centrifugal ultrafiltration member 122 to rotate. In some embodiments, the centrifugal ultrafiltration member 122 may be disposed in the processing chamber 11, and an ultrafiltration membrane layers 1221 may be arranged on each of two opposite sides of the centrifugal ultrafiltration member 122. A filtration chamber 1222 may be defined in the centrifugal ultrafiltration member 122. A waste liquid hole 1223 may be defined on the centrifugal ultrafiltration member 122. The waste liquid hole 1223 may be in communication with the filtration chamber 1222. The waste liquid hole 1223 may be further in communication with the discharge outlet 111.

From the above-mentioned structure, it may be seen that after collagen extraction solution/liquid enters the processing chamber 11, a small-molecule impurity in the collagen extraction solution may be filtered by the ultrafiltration membrane layer 1221. The impurity may enter an inner side of filtration chamber 1222 from an outer side of the ultrafiltration membrane layer 1221, and then the impurity may be discharged from the discharge outlet 111 via the waste liquid hole 1223, while the collagen molecular structure may be retained in the processing chamber 11. In some embodiments, a rotating structure may be utilized by the collagen processing structure 10 in the embodiments of the present disclosure, and the rotating structure may be completely different from cross-flow ultrafiltration in the related art. By combining with centrifugation and cross-flow filtration principles, on the one hand, the centrifugal driving member 121 may be configured to drive the centrifugal ultrafiltration member 122 to rotate, fluid may be separated into high-concentration fluid and low-concentration fluid by a centrifugal force. Different shear forces may be provided via different linear velocities between an inner circle and an outer circle to process feed liquid, thereby improving the ultrafiltration efficiency. In addition, a certain surface flow velocity may be formed on a surface of the centrifugal ultrafiltration member 122 via centrifugal motion, such that the cross-flow filtration may be implemented due to the different linear velocities between the inner circle and the outer circle. In this way, it may be possible to enable a substance to pass through and to be filtered out, where a size of the substance may be smaller than a membrane pore size of the ultrafiltration membrane layer 1221, and thus a collagen molecule may be retained, where a size of the collagen molecule may be larger than the membrane pore size of the ultrafiltration membrane layer 1221, thereby implementing the ultrafiltration purification of the collagen. On the other hand, the centrifugal force may be generated by a rotational motion of the centrifugal ultrafiltration member 122, and a strong/intense turbulence may be generated/arose from the different linear velocities in the collagen extraction solution during ultrafiltration, such that it may be possible to improve the efficiency of removing the filter layer on the membrane surface of the ultrafiltration membrane layer 1221, eliminate/reduce a concentration difference on the membrane surface of the ultrafiltration membrane layer 1221, and reduce an occurrence of membrane-blocking phenomena, thereby improving the collagen purity and the ultrafiltration efficiency.

In addition, the collagen extraction solution may be separated into the high-concentration fluid and the low-concentration fluid by utilizing the centrifugal force in some embodiments of the present disclosure, which may be a simple technical solution. Compared with the related art, the technical solution provided by some embodiments of the present disclosure does not rely on a high-volume recirculation flow rate and an operating pressure typically by adjusting the pressure. Therefore, a shear force of the contact between the centrifugal ultrafiltration member 122 and the collagen extraction solution, i.e., the shear force exerted by the centrifugal ultrafiltration assembly 122 on the collagen extraction solution, may be relatively weak, such that it may be conducive to protecting a collagen structure.

In some embodiments, the ultrafiltration membrane layer 1221 may be made of an inorganic ceramic material. The inorganic ceramic material may have strong resistance to acids, alkalis, and heat, and may be easy to clean and maintain, such that it may be possible to extend a service cycle of the ultrafiltration membrane layer 1221 and reduce pollution. In some embodiments, the ultrafiltration membrane layer 1221 may also be made of other materials, which is not be repeated herein.

In some embodiments, the centrifugal driving member 121 may include a hollow centrifugal shaft 1211 extending into the processing chamber 11. A hollow channel 1212 may be defined on the centrifugal shaft 1211, and an end of the centrifugal shaft 1211 away from the centrifugal driving member 121 may be connected to the discharge outlet 111. A mounting hole 1224 may be defined on each centrifugal ultrafiltration member 122. The centrifugal shaft 1211 may be fixed in the mounting hole 1224. The waste liquid hole 1223 may penetrate an inner wall of the mounting hole 1224. A liquid inlet (not shown) may be defined on the centrifugal shaft 1211. The liquid inlet may be in communication with the waste liquid hole 1223. Via the above-mentioned structure, the centrifugal shaft 1211 may be driven to rotate, such that the centrifugal ultrafiltration member 122 may be driven to rotate.

In an embodiment, the number of centrifugal ultrafiltration members 122 may be several. The several centrifugal ultrafiltration members 122 may be arranged at intervals on the centrifugal shaft 1211. The several centrifugal ultrafiltration members 122 may be driven to rotate via the centrifugal shaft 1211, such that a cross-flow fluid (or cross-current flow) may be formed, thereby performing cross-flow filtration on the collagen extraction solution. In addition, under the action of the centrifugal force, a filtered liquid may move from an end of the filtration chamber 1222 away from the centrifugal shaft 1211 to the hollow channel 1212 and enter the hollow channel 1212 for discharge, thereby improving the ultrafiltration efficiency. The each centrifugal ultrafiltration member 122 may be installed on the centrifugal shaft 1211 via the mounting hole 1224, and the filtration chamber 1222 may be in communication with the hollow channel 1212, such that it may be possible to discharge the impurity in the collagen extraction solution. That is, the impurity filtered by the ultrafiltration membrane layer 1221 may enter the filtration chamber 1222, and then enter the hollow channel 1212 via the waste liquid hole 1223 and the liquid inlet, and may be finally discharged via the discharge outlet 111, such that it may be possible to implement the purification of collagen in the collagen processing structure 10, thereby retaining the collagen molecular structure, discharging the remaining impurity, and improving the collagen purity.

In some embodiments, each mounting hole 1224 may be disposed at a center of a corresponding one of the several centrifugal ultrafiltration members 122, such that it may be possible to reduce a rotational radius of the centrifugal ultrafiltration member 122 around the centrifugal shaft 1211. That is, the centrifugal ultrafiltration member 122 may rotate with a radius, where the radius may be a distance from the center of the centrifugal ultrafiltration member 122 to an edge of the centrifugal ultrafiltration member 122, such that it may be possible to reduce space occupied by the centrifugal ultrafiltration member 122 and save structural costs.

In some embodiments, the each centrifugal ultrafiltration member 122 may be disc-shaped, i.e., in a shape of a disc. The ultrafiltration membrane layer 1221 may be arranged on each of two opposite sides of the disc, and the mounting hole 1224 may be a center of the disc. That is, the centrifugal ultrafiltration member 122 may rotate around the centrifugal shaft 1211 with the radius of the centrifugal ultrafiltration member 122, so as to improve the ultrafiltration efficiency.

In some embodiments, a feed inlet 112 and a liquid return port 113 may be defined on the processing chamber 11. Along a direction of gravity, each of the discharge outlet 111 and the feed inlet 112 may be disposed at a bottom of the liquid return port 113. The collagen extraction solution may enter the processing chamber 11 from the feed inlet 112. On the one hand, a collagen structure after ultrafiltration may enter the liquid return port 113 in an opposite direction of gravity. On the other hand, the filtered impurity may enter the filtration chamber 1222 and may be discharged along the direction of gravity via the discharge outlet 111, thereby enabling more thorough ultrafiltration of the collagen.

In some embodiments, the membrane pore size of the each ultrafiltration membrane layer 1221 may be in a range from 10 nm to 30 nm, such as 10 nm, 15 nm, 20 nm, 25 nm, 30 nm, etc. in this way, it may be possible to ultrafilter the collagen extraction solution and separate the collagen solution from small-molecule impurity and other substances.

As shown in FIG. 4, FIG. 4 is a schematic structural view of a collagen processing system according to some embodiments of the present disclosure. According to another aspect, a collagen processing system 100 may be provided by some embodiments of the present disclosure. The collagen processing system 100 may include the collagen processing structure 10 described in any one of above-mentioned embodiments, a storage tank 20, a delivering pipeline 30, a return pipeline 40, a sewage pipeline 50, and a water injection pipeline 60. The delivering pipeline 30 may be connected to the storage tank 20 and the collagen processing structure 10. The return pipeline 40 may be connected to the collagen processing structure 10 and the storage tank 20. The sewage pipeline 50 may be connected to the discharge outlet 111. The water injection pipeline 60 may be connected to the storage tank 20. In some embodiments, the collagen processing system 100 may further be arranged with a pressure-regulating assembly 70. The pressure-regulating assembly 70 may include a compressed air inlet 71 and an ultrafiltration pump 72. The compressed air inlet 71 may be connected to the storage tank 20 and may be configured to adjust a pressure in the storage tank 20. One end of the ultrafiltration pump 72 may be connected to the storage tank 20, and another end of the ultrafiltration pump 72 may be connected to the collagen processing structure 10. The ultrafiltration pump 72 may be configured to provide power to deliver a material in the storage tank 20 to the collagen processing structure 10.

In some embodiments, the storage tank 20 may be configured to store the material, such as water, collagen extraction solution, and collagen solution after ultrafiltration. The delivering pipeline 30 may be configured to deliver the materials in the storage tank 20 to the collagen processing structure 10. In some embodiments, the ultrafiltration pump 72 may be arranged on the delivering pipeline 30. One end of the ultrafiltration pump 72 may be connected to the storage tank 20, and another end of the ultrafiltration pump 72 may be connected to the collagen processing structure 10. The ultrafiltration pump 72 may be configured to adjust the pressure in the storage tank 20 and a pressure in the collagen processing structure 10, such that it may be possible to ensure that the liquid may be smoothly output. The return pipeline 40 may be configured to return the collagen solution after ultrafiltration performed by the centrifugal processing mechanism 12 to the storage tank 20, so as to perform cyclic ultrafiltration. In some embodiments, the pressure-regulating assembly 70 may further include a return pump arranged on the return pipeline 40, and the return pump may be configured to adjust a return pressure, so as to ensure the liquid to be returned. In some embodiments, the pressure-regulating assembly may also be arranged in other positions, so as to increase or decrease the pressure according to different stages. The sewage pipeline 50 may be configured to discharge the filtered impurity. The water injection pipeline 60 may be configured to inject the water into the storage tank 20, so as to maintain material balance in the storage tank 20. It should be noted that an entire processing process of the collagen processing system 100 may include two stages. In some embodiments, the two stages may include a purification stage and a concentration stage.

In the purification stage, initial collagen extraction solution in the storage tank 20 may be delivered to the collagen processing structure 10. The centrifugal processing mechanism 12 of the collagen processing structure 10 may perform centrifugal ultrafiltration on the initial collagen extraction solution. The collagen solution after ultrafiltration may be returned to the storage tank 20 via the return pipeline 40, and the filtrated waste liquid including the impurity may be discharged via the sewage pipeline 50. According to amount of discharged waste liquid, equal amount of injection water may be injected into the storage tank 20 via the water injection pipeline 60, and the cyclic ultrafiltration may continue to be performed until the purification is completed. After the purification is completed, the concentration may be directly carried out without replacing an equipment, adding a concentration reagent, or the like, and the operation may be simple.

In the concentration stage, the water injection pipeline 60 may be closed, the collagen solution in the storage tank 20 may continue to be delivered to the collagen processing structure 10, the collagen after ultrafiltration may be returned to the storage tank 20 via the return pipeline 40, and filtrate may be discharged via the sewage pipeline 50, which may be repeated until the concentration is completed.

In an embodiment, the water injection pipeline 60 may be controlled to be in a working state of closing water injection or another working state of injecting water into the storage tank 20. In some embodiments, the water injection pipeline 60 may be controlled to be in the working state of closing water injection for a next-stage operation. For example, after the purification is completed, the water injection may be closed for concentration, etc. In some embodiments, the water injection pipeline 60 may further be controlled to be in the another working state of injecting water into the storage tank 20. At this time, a flow meter 61 may be arranged on each of the water injection pipeline 60 and the sewage pipeline 50, such that it may be possible to calculate the liquid flow rate, and thus amount of water injected into the storage tank 20 may be equal to amount of waste liquid discharged from the discharge outlet 111, thereby maintaining the material balance in the storage tank 20. In some embodiments, the water may be automatically injected into the storage tank 20 according to the amount of the waste liquid discharged from the discharge outlet 111, thereby achieving automation.

In some embodiments, the collagen processing system 100 may further include a flushing tank 80 and a flushing pipeline 90. The flushing pipeline 90 may be connected to the collagen processing structure 10 and the flushing tank 80. A cleaning agent may be injected into the flushing tank 80, and the cleaning agent may be delivered to the collagen processing structure 10 via the flushing pipeline 90, so as to clean the collagen processing structure 10. In some embodiments, the pressure-regulating assembly 70 may further include a flushing pump 73. The flushing pump 73 may be arranged on the flushing pipeline 90. The flushing pump 73 may be configured to provide power to deliver the cleaning reagent in the flushing tank 80 to the collagen processing structure 10 and adjust the pressure in the flushing pipeline 90. A back-flushing principle may be applied in the above-mentioned technical scheme, such that it may be possible to facilitate cleaning the system, extend the service cycle of the membrane, and reduce the membrane pollution phenomenon.

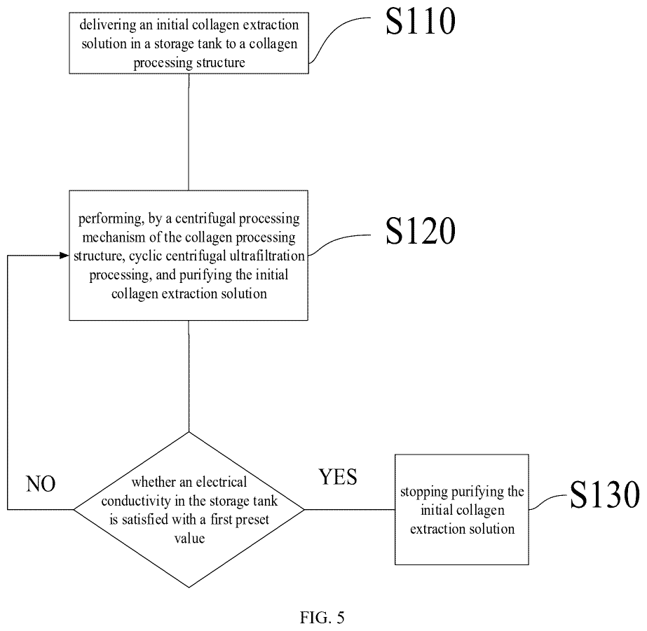

As shown in FIG. 5, FIG. 5 is a schematic diagram of a purification process of a method for processing collagen according to some embodiments of the present disclosure. According to another aspect, a method for processing collagen may be provided by some embodiments of the present disclosure. The method for processing collagen may include the following operations.

At block S110, initial collagen extraction solution in a storage tank 20 may be delivered to a collagen processing structure 10.

In some embodiments, pressure may be adjusted by an ultrafiltration pump 72 of a pressure-regulating assembly 70. Feed pressure for the initial collagen extraction solution to enter the collagen processing structure 10 may be in a range from 0.1 MPa to 0.3 MPa, such as 0.1 MPa, 0.2 MPa, and 0.3 MPa, etc., so as to ensure that the initial collagen extraction solution may be smoothly delivered to the collagen processing structure 10.

At block S120, cyclic centrifugal ultrafiltration processing may be performed by a centrifugal processing mechanism 12 of the collagen processing structure 10, and the initial collagen extraction solution may be purified.

In some embodiments, during a process of performing, by the collagen processing structure 10, the cyclic centrifugal ultrafiltration processing, and purifying the initial collagen extraction solution, a rotation speed of the centrifugal processing mechanism 12 may be in a range from 100 rpm to 500 rpm, such as 200 rpm, 300 rpm, 400 rpm, and 500 rpm, etc., so as to improve the ultrafiltration efficiency. Discharge pressure of the processing chamber 11 may be in a range from 0.01 MPa to 0.3 MPa, such as 0.01 MPa, 0.05 MPa, 0.10 MPa, 0.15 MPa, 0.20 MPa, 0.25 MPa, and 0.30 MPa, etc., so as to ensure that collagen extraction solution after ultrafiltration may be smoothly output.

In some embodiments, an operation of performing, by the collagen processing structure 10, the cyclic centrifugal ultrafiltration processing, and purifying the initial collagen extraction solution may include the following processes, may include: discharging waste liquid generated in each centrifugal ultrafiltration processing; returning collagen liquid extracted in the each centrifugal ultrafiltration processing to the storage tank 20, and simultaneously supplementing the same amount of water as the waste liquid into the storage tank 20; delivering mixed liquid to the collagen processing structure 10 again and performing another centrifugal ultrafiltration processing, and repeating until the purifying the initial collagen extraction solution is stopped, where the mixed liquid may be formed by formed by remaining initial collagen extraction solution in the storage tank 20, supplemented water, and the collagen liquid extracted in the centrifugal ultrafiltration processing. In some embodiments, waste liquid, which may include impurity and may be generated during the centrifugal ultrafiltration processing, may enter the filtration chamber 1222. In addition, under an action of a centrifugal force, the waste liquid may enter the hollow channel 1212 via the waste liquid hole 1223 and the liquid inlet, and may be discharged via the sewage pipeline 50. The collagen molecular structure after ultrafiltration may be retained in the processing chamber 11 and mixed with liquid, and may return to the storage tank 20. The waste liquid including impurity may be discharged via the sewage pipeline 50. In order to maintain material balance in the storage tank 20, certain amount of waste liquid may be discharged, that is, certain amount of injection water may be injected.

At block S130, in a case where an electrical conductivity in the storage tank 20 may be satisfied with a first preset value, the initial collagen extraction solution may be stopped purifying.

In some embodiments, in a case where the electrical conductivity in the storage tank 20 may be satisfied with the first preset value, purification may be completed. In some embodiments, the first preset value may be set to 50 μS/cm, that is, when the electrical conductivity of the collagen liquid is less than or equal to 50 μS/cm, the purification may be completed.

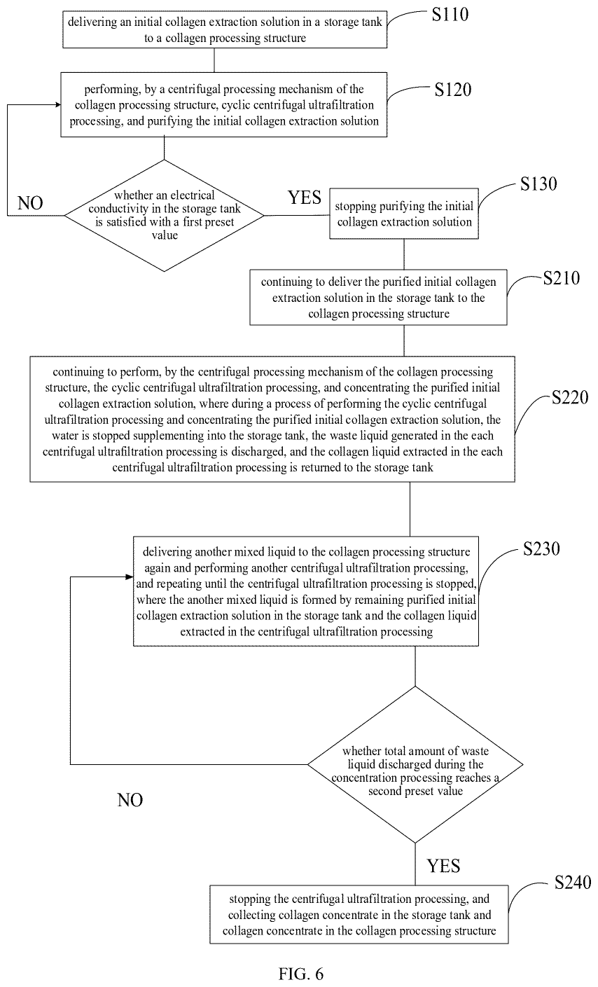

As shown in FIG. 4 and FIG. 6, FIG. 6 is a schematic diagram of a concentration process of the method for processing collagen according to some embodiments of the present disclosure. In some embodiments, after stopping purifying the initial collagen extraction solution, the method for processing collagen may further include performing a concentration processing on purified initial collagen extraction solution. An operation of performing the concentration processing may include the following operations.

At block S210, the purified initial collagen extraction solution in the storage tank 20 may be continued to deliver to the collagen processing structure 10.

In some embodiments, in order to concentrate the collagen liquid and increase the concentration of collagen, the purified initial collagen extraction solution in the storage tank 20 may be continuously delivered to the collagen processing structure 10 for ultrafiltration processing. Feed pressure of the processing chamber 11 may be in a range from 0.1 MPa to 0.5 MPa, such as 0.1 MPa, 0.2 MPa, 0.3 MPa, 0.4 MPa, 0.5 MPa, etc., so as to ensure that the purified initial collagen extraction solution may be smoothly delivered to the collagen processing structure 10.

At block S220, cyclic centrifugal ultrafiltration processing may be performed by the centrifugal processing mechanism 12 of the collagen processing structure 10, and the purified initial collagen extraction solution may be concentrated. During a process of performing the cyclic centrifugal ultrafiltration processing and concentrating the purified initial collagen extraction solution, the water may be stopped supplementing into the storage tank 20, the waste liquid generated in the each centrifugal ultrafiltration processing may be discharged, and the collagen liquid extracted in the each centrifugal ultrafiltration processing may be returned to the storage tank 20.

In some embodiments, the purified initial collagen extraction solution may be further concentrated by stopping supplementing the water into the storage tank 20, so as to increase the collagen concentration. During a process of concentrating the purified initial collagen extraction solution, a rotation speed of the centrifugal processing mechanism 12 may be in a range from 500 rpm to 800 rpm, such as 500 rpm, 600 rpm, 700 rpm, 800 rpm, etc., and discharge pressure of the processing chamber 11 may be in a range from 0.05 MPa to 0.5 MPa, such as 0.05 MPa, 0.15 MPa, 0.25 MPa, 0.35 MPa, 0.45 MPa, 0.5 MPa, etc. In this way, it may possible to improve the ultrafiltration efficiency, reduce the shear force, and protect the collagen molecular structure.

At block S230, delivering another mixed liquid to the collagen processing structure 10 again and performing another centrifugal ultrafiltration processing, and repeating until the centrifugal ultrafiltration processing is stopped, where the another mixed liquid is formed by remaining purified initial collagen extraction solution in the storage tank 20 and the collagen liquid extracted in the centrifugal ultrafiltration processing.

In some embodiments, the cyclic ultrafiltration may be continuously performed for further concentration processing on the mixed liquid, which may be formed by the remaining purified initial collagen extraction solution in the storage tank 20 and the collagen liquid extracted in the centrifugal ultrafiltration processing, such that the collagen concentration may be increased.

At block S240, in a case where total amount of waste liquid discharged during the concentration processing reaches a second preset value, the centrifugal ultrafiltration processing may be stopped, and collagen concentrate in the storage tank 20 and collagen concentrate in the collagen processing structure 10 may be collected.

In some embodiments, when total amount of the waste liquid discharged during the concentration processing reaches the second preset value, the concentration may be completed, the centrifugal ultrafiltration processing may be stopped, and the collagen concentrate in the storage tank 20 and remaining collagen concentrate in the collagen processing structure 10 may be collected. The second preset value may be set according to total amount of the initial collagen extraction solution, so as to meet the requirements for the concentrated collagen concentration.

In some embodiments, an operation of collecting the collagen concentrate in the storage tank 20 and the collagen concentrate in the collagen processing structure 10 may include: adjusting pressure in the storage tank 20 to be in a range from 0.2 MPa to 0.3 MPa, collecting the collagen concentrate from a first discharge outlet 21 of the storage tank 20, and closing the first discharge outlet 21 after completing collecting; and adjusting the centrifugal processing mechanism 12 to rotate at a speed of 200 rpm-300 rpm, and opening a second discharge outlet 13 of a processing chamber 11 to collect the collagen concentrate.

In some embodiments, pressure in the storage tank 20 may be adjusted by opening a compressed air inlet 71 connected to the storage tank 20. The pressure in the storage tank 20 may be adjusted to be in a range from 0.2 MPa to 0.3 MPa, such as 0.2 MPa, 0.22 MPa, 0.24 MPa, 0.26 MPa, 0.28 MPa, 0.3 MPa, etc. Along a direction of gravity, the first discharge outlet 21 may be disposed at a bottom of the compressed air inlet 71, such that the collagen concentrate may be discharged from the first discharge outlet 21 under a dual action of the pressure and the gravity, thereby improving a recovery rate of collagen. A rotation speed of the centrifugal driving member 121 may be adjusted to be in a range from 200 rpm to 300 rpm, such as 200 rpm, 220 rpm, 240 rpm, 260 rpm, 280 rpm, 300 rpm, etc., such that it may be possible to reduce the collagen residue on the centrifugal ultrafiltration member 122 under the action of centrifugal force, thereby improving the recovery efficiency of the collagen concentrate.

In the collagen processing system 100 of the embodiments of the present disclosure, the concentration processing may be directly performed after the purification of the collagen extraction solution is completed, and it is not necessary to replace the equipment or add a reagent, so as to simplify the operation, and thus the technology may be simple. Moreover, it may be possible to increase the collagen concentration of the collagen concentrate. In some embodiments, the collagen concentration may be greater than or equal to 20 mg/ml. It should be noted that the collagen processing system 100 of the embodiments of the present disclosure may be aimed at purifying and concentrating collagen with characteristics such as the high viscosity, the poor water solubility, and the tendency to gelation, so as to obtain the collagen with a high concentration multiple that may be concentrated to more than 20 mg/ml. in this way, it may be possible to effectively remove non-collagen impurity, have a high yield, and a complete triple-helix structure, thereby meeting the requirement of higher-concentration collagen medical materials.

In some embodiments, after collecting the collagen concentrate in the storage tank 20 and the collagen concentrate in the collagen processing structure 10, the method for processing collagen may further include: flushing the collagen processing structure 10. That is, after collecting the collagen concentrate, the collagen processing structure 10 may be flushed by the flushing tank 80 and the flushing pipeline 90, so as to extend the service cycle of the centrifugal ultrafiltration member 122 and reduce the pollution phenomenon.

In some embodiments, an operation of flushing the collagen processing structure 10 may include: closing a delivering pipeline 30 between the collagen processing structure 10 and the storage tank 20, and closing a discharge outlet 111 of the processing chamber 11; connecting a flushing pipeline 90 to the collagen processing structure 10; adjusting inlet pressure of the discharge outlet 111 of the processing chamber 11 to be in a range from 0.1 MPa to 0.2 MPa, and adjusting the rotation speed of the centrifugal driving member 121 to be in a range from 200 rpm to 300 rpm; and performing cleaning.

In some embodiments, by closing the delivering pipeline 30 and the discharge outlet 111, it may be ensured that all openings may be closed, and the all openings may be disposed at a bottom of the liquid return port 113 and in the collagen processing structure 10 along the direction of gravity, such that the cleaning agent may flow unidirectionally, thereby improving the cleaning efficiency. The cleaning agent may be delivered to the collagen processing structure 10 via the flushing pipeline 90. In order to improve the delivering efficiency and ensure that the cleaning agent may be smoothly delivered to the collagen processing structure 10, a flushing pump 73 may be arranged to adjust feed pressure of the processing chamber 11. The pressure may be adjusted to be in a range from 0.1 MPa to 0.2 MPa, such as 0.1 MPa, 0.12 MPa, 0.14 MPa, 0.16 MPa, 0.18 MPa, 0.2 MPa, etc. The rotation speed of the centrifugal driving member 121 may be adjusted to be in a range from 200 rpm to 300 rpm, such as 200 rpm, 220 rpm, 240 rpm, 260 rpm, 280 rpm, 300 rpm, etc., such that the centrifugal ultrafiltration member 122 may be fully contacted with the cleaning agent, thereby increasing the cleaning area and improving the cleaning efficiency. In some embodiments, the flushing tank 80 may be connected to the cleaning agent injection pipeline 81. The cleaning agent may be injected via the cleaning agent injection pipeline 81, and the cleaning agent may be delivered to the collagen processing structure 10 via the flushing pipeline 90 to clean the processing chamber 11 and the centrifugal processing mechanism 12. Then, the cleaning agent may return to the storage tank 20 via the return pipeline 40 and may be finally discharged via the first discharge outlet 21 of the storage tank 20.

In the method for processing collagen of the embodiments of the present disclosure, the purification process, the concentration process, and the subsequent cleaning process may be completed on the same set of systems, with simple operation. The following four tests may show experimental data of the collagen processing structure, the collagen processing system, and the method for processing collagen in the embodiments of the present disclosure:

A first test may include the following operations.

In an operation 1, the collagen extraction solution may be placed in the storage tank 20 of the collagen processing system 100. The concentration of the collagen extraction solution may be 2.2 mg/ml, a viscosity may be 126 mPa·s, and amount of material may be 50 kg.

In an operation 2, a pore size of the centrifugal ultrafiltration member 122 may be 10 nm. Parameters of the centrifugal ultrafiltration member 122 during the purification process may be set as follows. The feed pressure may be 0.1 MPa. The rotation speed of the centrifugal ultrafiltration member 122 may be 300 rpm. The discharge pressure may be 0.08 MPa. Continuous replenishment of injection water may be started.

In an operation 3, in a case where the electrical conductivity of the collagen purified solution in the storage tank 20 is less than or equal to 50 μS/cm, the concentration process may be started.

In an operation 4, parameters the centrifugal ultrafiltration member 122 during the concentration process may be set as follows. The feed pressure may be 0.2 MPa. The rotation speed of the centrifugal ultrafiltration member 122 may be 500 rpm. The discharge pressure may be 0.18 MPa. The replenishment of injection water may be stopped.

In an operation 5, after the concentration is completed, the compressed air inlet 71 may be opened, the pressure in the storage tank 20 may be set to be 0.2 MPa, the collagen concentrate may be collected at the first discharge outlet 21 of the storage tank 20. After the material in the storage tank 20 is collected, the first discharge outlet 21 may be closed, the rotation speed of the centrifugal ultrafiltration member may be set to be 200 rpm, and the second discharge outlet 13 of the processing chamber 11 may be opened to collect the collagen concentrate.

In an operation 6, cleaning may be performed after the material collection is completed.

Finally, the purification may take 41 minutes, the concentration may take 27 minutes, 6.44 kg of collagen concentrate may be collected, the collagen concentration may be 15.7 mg/ml, the viscosity may be 18970 mPa·s, the yield may be 91.92%, and the concentration multiple may be 7.14.

A second test may include the following operations.

In an operation 1, the collagen extraction solution may be placed in the storage tank 20 of the collagen processing system 100. The concentration of the collagen extraction solution may be 3.5 mg/ml, a viscosity may be 184 mPa·s, and amount of material may be 42 kg.

In an operation 2, a pore size of the centrifugal ultrafiltration member 122 may be 30 nm. Parameters of the centrifugal ultrafiltration member 122 during the purification process may be set as follows. The feed pressure may be 0.05 MPa. The rotation speed of the centrifugal ultrafiltration member 122 may be 500 rpm. The discharge pressure may be 0.04 MPa. Continuous replenishment of injection water may be started.

In an operation 3, in a case where the electrical conductivity of the collagen purified solution in the storage tank 20 is less than or equal to 50 μS/cm, the concentration process may be started.

In an operation 4, parameters the centrifugal ultrafiltration member 122 during the concentration process may be set as follows. The feed pressure may be 0.15 MPa. The rotation speed of the centrifugal ultrafiltration member 122 may be 800 rpm. The discharge pressure may be 0.14 MPa. The replenishment of injection water may be stopped.

In an operation 5, after the concentration is completed, the compressed air inlet 71 may be opened, the pressure in the storage tank 20 may be set to be 0.15 MPa, the collagen concentrate may be collected at the first discharge outlet 21 of the storage tank 20. After the material in the storage tank 20 is collected, the first discharge outlet 21 may be closed, the rotation speed of the centrifugal ultrafiltration member may be set to be 300 rpm, and the second discharge outlet 13 of the processing chamber 11 may be opened to collect the collagen concentrate.

In an operation 6, cleaning may be performed after the material collection is completed.

Finally, the purification may take 34 minutes, the concentration may take 21 minutes, 6.89 kg of collagen concentrate may be collected, the collagen concentration may be 18.2 mg/ml, the viscosity may be 21550 mPa·s, the yield may be 85.30%, and the concentration multiple may be 5.20.

A third test may include the following operations.

In an operation 1, the collagen extraction solution may be placed in the storage tank 20 of the collagen processing system 100. The concentration of the collagen extraction solution may be 1.8 mg/ml, a viscosity may be 105 mPa·s, and amount of material may be 70 kg.

In an operation 2, a pore size of the centrifugal ultrafiltration member 122 may be 16 nm. Parameters of the centrifugal ultrafiltration member 122 during the purification process may be set as follows. The feed pressure may be 0.2 MPa. The rotation speed of the centrifugal ultrafiltration member 122 may be 400 rpm. The discharge pressure may be 0.17 MPa. Continuous replenishment of injection water may be started.

In an operation 3, in a case where the electrical conductivity of the collagen purified solution in the storage tank 20 is less than or equal to 50 μS/cm, the concentration process may be started.

In an operation 4, parameters the centrifugal ultrafiltration member 122 during the concentration process may be set as follows. The feed pressure may be 0.3 MPa. The rotation speed of the centrifugal ultrafiltration member 122 may be 600 rpm. The discharge pressure may be 0.29 MPa. The replenishment of injection water may be stopped.

In an operation 5, after the concentration is completed, the compressed air inlet 71 may be opened, the pressure in the storage tank 20 may be set to be 0.2 MPa, the collagen concentrate may be collected at the first discharge outlet 21 of the storage tank 20. After the material in the storage tank 20 is collected, the first discharge outlet 21 may be closed, the rotation speed of the centrifugal ultrafiltration member may be set to be 250 rpm, and the second discharge outlet 13 of the processing chamber 11 may be opened to collect the collagen concentrate.

In an operation 6, cleaning may be performed after the material collection is completed.

Finally, the purification may take 28 minutes, the concentration may take 37 minutes, 5.16 kg of collagen concentrate may be collected, the collagen concentration may be 21.7 mg/ml, the viscosity may be 26470 mPa·s, the yield may be 88.87%, and the concentration multiple may be 12.06.

A four test may include the following operations.

In an operation 1, the collagen extraction solution may be placed in the storage tank 20 of the collagen processing system 100. The concentration of the collagen extraction solution may be 4.1 mg/ml, a viscosity may be 230 mPa·s, and amount of material may be 35 kg.

In an operation 2, a pore size of the centrifugal ultrafiltration member 122 may be 24 nm. Parameters of the centrifugal ultrafiltration member 122 during the purification process may be set as follows. The feed pressure may be 0.3 MPa. The rotation speed of the centrifugal ultrafiltration member 122 may be 200 rpm. The discharge pressure may be 0.27 MPa. Continuous replenishment of injection water may be started.

In an operation 3, in a case where the electrical conductivity of the collagen purified solution in the storage tank 20 is less than or equal to 50 μS/cm, the concentration process may be started.

In an operation 4, parameters the centrifugal ultrafiltration member 122 during the concentration process may be set as follows. The feed pressure may be 0.5 MPa. The rotation speed of the centrifugal ultrafiltration member 122 may be 700 rpm. The discharge pressure may be 0.48 MPa. The replenishment of injection water may be stopped.

In an operation 5, after the concentration is completed, the compressed air inlet 71 may be opened, the pressure in the storage tank 20 may be set to be 0.25 MPa, the collagen concentrate may be collected at the first discharge outlet 21 of the storage tank 20. After the material in the storage tank 20 is collected, the first discharge outlet 21 may be closed, the rotation speed of the centrifugal ultrafiltration member may be set to be 280 rpm, and the second discharge outlet 13 of the processing chamber 11 may be opened to collect the collagen concentrate.

In an operation 6, cleaning may be performed after the material collection is completed.

Finally, the purification may take 47 minutes, the concentration may take 33 minutes, 5.46 kg of collagen concentrate may be collected, the collagen concentration may be 23.1 mg/ml, the viscosity may be 32280 mPa·s, the yield may be 87.89%, and the concentration multiple may be 5.63.

It may be seen from the above data that the collagen processing structure, the collagen processing system, and the method for processing collagen in the embodiments of the present disclosure may have simple operation and technology. After the purification and concentration of the collagen extraction solution, the collagen concentration may be high, the time used may be short, such that the ultrafiltration efficiency may be improved, and the recovery rate of collagen may be increased.

Finally, it should be noted that, the above-mentioned embodiments are only used to illustrate the technical solutions of the present disclosure, not to limit them. Although the present disclosure has been described in detail with reference to the foregoing embodiments, those of ordinary skill in the art should understand that they can still modify the technical solutions described in the foregoing embodiments, or equivalently replace some or all of the technical features. These modifications or replacements do not make the essence of the corresponding technical solutions deviate from the scope of the technical solutions of the embodiments of the present disclosure, and they should all be covered within the scope of the claims and the specification of the present disclosure. In particular, as long as there may be no structural conflict, the technical features mentioned in each embodiment may be combined in any way. The present disclosure may be not limited to the specific embodiments disclosed herein, but may include all technical solutions falling within the scope of the claims.

1. A collagen processing structure, comprising:
a processing chamber, defining a discharge outlet; and
a centrifugal processing mechanism, comprising a centrifugal driving member and a centrifugal ultrafiltration member, wherein a centrifugal ultrafiltration member is connected to the centrifugal driving member, and the centrifugal driving member is configured to drive the centrifugal ultrafiltration member to rotate;
wherein the centrifugal ultrafiltration member is disposed in the processing chamber, and an ultrafiltration membrane layer is arranged on each of two opposite sides of the centrifugal ultrafiltration member, a filtration chamber is defined in the centrifugal ultrafiltration member, a waste liquid hole is defined on the centrifugal ultrafiltration member and is in communication with the filtration chamber, and the waste liquid hole is further in communication with the discharge outlet.

2. The collagen processing structure according to claim 1, wherein the centrifugal driving member comprises a hollow centrifugal shaft extending into the processing chamber, a hollow channel is defined on the centrifugal shaft, and an end of the centrifugal shaft away from the centrifugal driving member is connected to the discharge outlet;
the number of centrifugal ultrafiltration members is one or more, a mounting hole is defined on each of the one or more centrifugal ultrafiltration members, and the centrifugal shaft is fixed in the mounting hole;
the waste liquid hole penetrates an inner wall of the mounting hole; and
a liquid inlet is defined on the centrifugal shaft and is in communication with the waste liquid hole.

3. The collagen processing structure according to claim 2, wherein each mounting hole is disposed at a center of a corresponding one of the one or more centrifugal ultrafiltration members.

4. The collagen processing structure according to claim 3, wherein the each of the one or more centrifugal ultrafiltration members is in a shape of a disc, the ultrafiltration membrane layer is arranged on each of two opposite sides of the disc, and the mounting hole is a center of the disc.

5. The collagen processing structure according to claim 4, wherein the processing chamber further defines a feed inlet and a liquid return port, and along a direction of gravity, each of the discharge outlet and the feed inlet are disposed at a bottom of the liquid return port.

6. The collagen processing structure according to claim 1, wherein a membrane pore size of the each ultrafiltration membrane layer is in a range from 10 nm to 30 nm.

7. The collagen processing structure according to claim 1, wherein the number of centrifugal ultrafiltration members is several, the several centrifugal ultrafiltration members are arranged at intervals on the centrifugal shaft, and the several centrifugal ultrafiltration members are driven to rotate via the centrifugal shaft.

8. The collagen processing structure according to claim 1, wherein the ultrafiltration membrane layer is made of an inorganic ceramic material.

9. A collagen processing system, comprising a collagen processing structure;
wherein the collagen processing system further comprises a storage tank, a delivering pipeline, a return pipeline, a sewage pipeline, and a water injection pipeline;
the delivering pipeline is connected to the storage tank and the collagen processing structure;
the return pipeline is connected to the collagen processing structure and the storage tank;
the sewage pipeline is connected to the discharge outlet; and
the water injection pipeline is connected to the storage tank;
wherein the collagen processing structure comprises:
a processing chamber, defining a discharge outlet; and
a centrifugal processing mechanism, comprising a centrifugal driving member and one or more centrifugal ultrafiltration members, wherein a corresponding one of the one or more centrifugal ultrafiltration members is connected to the centrifugal driving member, and the centrifugal driving member is configured to drive the corresponding one of the one or more centrifugal ultrafiltration members to rotate;
wherein the corresponding one of the one or more centrifugal ultrafiltration members is disposed in the processing chamber, and an ultrafiltration membrane layer is arranged on each of two opposite sides of the corresponding one of the one or more centrifugal ultrafiltration members, a filtration chamber is defined in the corresponding one of the one or more centrifugal ultrafiltration members, a waste liquid hole is defined on the corresponding one of the one or more centrifugal ultrafiltration members and is in communication with the filtration chamber, and the waste liquid hole is further in communication with the discharge outlet.

10. The collagen processing system according to claim 9, wherein the water injection pipeline is controlled to be in a working state of closing water injection or another working state of injecting water into the storage tank; and
in the another working state of injecting water into the storage tank, amount of water injected into the storage tank is equal to amount of waste liquid discharged from the discharge outlet.

11. The collagen processing system according to claim 9, further comprising a pressure-regulating assembly, wherein the pressure-regulating assembly comprises a compressed air inlet and an ultrafiltration pump;
the compressed air inlet is connected to the storage tank and is configured to adjust a pressure in the storage tank; and
one end of the ultrafiltration pump is connected to the storage tank, and another end of the ultrafiltration pump is connected to the collagen processing structure.

12. The collagen processing system according to claim 9, further comprising a flushing tank, a flushing pump and a flushing pipeline, wherein the flushing pipeline is connected to the collagen processing structure and the flushing tank.

13. The collagen processing system according to claim 9, wherein the water injection pipeline is controlled to be in a working state of injecting water into the storage tank, and a flow meter is arranged on each of the water injection pipeline and the sewage pipeline.

14. A method for processing collagen, comprising:
delivering an initial collagen extraction solution in a storage tank to a collagen processing structure;
performing, by a centrifugal processing mechanism of the collagen processing structure, cyclic centrifugal ultrafiltration processing, and purifying the initial collagen extraction solution; and
in a case where an electrical conductivity in the storage tank is satisfied with a first preset value, stopping purifying the initial collagen extraction solution;
wherein performing, by the centrifugal processing mechanism of the collagen processing structure, the cyclic centrifugal ultrafiltration processing, and purifying the initial collagen extraction solution, comprises:
discharging waste liquid generated in each centrifugal ultrafiltration processing, returning collagen liquid extracted in the each centrifugal ultrafiltration processing to the storage tank, and simultaneously supplementing the same amount of water as the waste liquid into the storage tank;
delivering mixed liquid to the collagen processing structure again and performing another centrifugal ultrafiltration processing, and repeating until the purifying the initial collagen extraction solution is stopped, wherein the mixed liquid is formed by remaining initial collagen extraction solution in the storage tank, supplemented water, and the collagen liquid extracted in the centrifugal ultrafiltration processing.

15. The method for processing collagen according to claim 14, wherein after stopping purifying the initial collagen extraction solution, the method further comprises performing a concentration processing on purified initial collagen extraction solution;
wherein performing the concentration processing on the purified initial collagen extraction solution, comprises:
continuing to deliver the purified initial collagen extraction solution in the storage tank to the collagen processing structure; and
continuing to perform, by the centrifugal processing mechanism of the collagen processing structure, the cyclic centrifugal ultrafiltration processing, and concentrating the purified initial collagen extraction solution, wherein during a process of performing the cyclic centrifugal ultrafiltration processing and concentrating the purified initial collagen extraction solution, the water is stopped supplementing into the storage tank, the waste liquid generated in the each centrifugal ultrafiltration processing is discharged, and the collagen liquid extracted in the each centrifugal ultrafiltration processing is returned to the storage tank;
delivering another mixed liquid to the collagen processing structure again and performing another centrifugal ultrafiltration processing, and repeating until the centrifugal ultrafiltration processing is stopped, wherein the another mixed liquid is formed by remaining purified initial collagen extraction solution in the storage tank and the collagen liquid extracted in the centrifugal ultrafiltration processing; and
in a case where total amount of waste liquid discharged during the concentration processing reaches a second preset value, stopping the centrifugal ultrafiltration processing, and collecting collagen concentrate in the storage tank and collagen concentrate in the collagen processing structure.

16. The method for processing collagen according to claim 14, wherein during a process of performing, by the collagen processing structure, the cyclic centrifugal ultrafiltration processing, and purifying the initial collagen extraction solution, a rotation speed of the centrifugal processing mechanism is in a range from 100 rpm to 500 rpm, feed pressure of the processing chamber is in a range from 0.1 MPa to 0.3 MPa, and discharge pressure of the processing chamber is in a range from 0.01 MPa to 0.3 MPa.

17. The method for processing collagen according to claim 14, wherein during a process of performing the concentration processing on the purified initial collagen extraction solution, a rotation speed of the centrifugal processing mechanism is in a range from 500 rpm to 800 rpm, feed pressure of the processing chamber is in a range from 0.1 MPa to 0.5 MPa, and discharge pressure of the processing chamber is in a range from 0.05 MPa to 0.5 MPa.

18. The method for processing collagen according to claim 14, wherein collecting the collagen concentrate in the storage tank and the collagen concentrate in the collagen processing structure, comprises:
adjusting pressure in the storage tank to be in a range from 0.2 MPa to 0.3 MPa, collecting the collagen concentrate from a first discharge outlet of the storage tank, and closing the first discharge outlet after completing collecting; and
adjusting the centrifugal processing mechanism to rotate at a speed of 200 rpm-300 rpm, and opening a second discharge outlet of a processing chamber to collect the collagen concentrate.

19. The method for processing collagen according to claim 14, wherein after collecting the collagen concentrate in the storage tank and the collagen concentrate in the collagen processing structure, the method further comprises: flushing the collagen processing structure;
wherein flushing the collagen processing structure, comprises:
closing a delivering pipeline between the collagen processing structure and the storage tank, and closing a sewage pipeline;
connecting a flushing pipeline to the collagen processing structure; and
starting a flushing pump on the flushing pipeline, adjusting inlet pressure of a discharge outlet of the processing chamber to be in a range from 0.1 MPa to 0.2 MPa, and adjusting a rotation speed of the centrifugal processing mechanism to be in a range from 200 rpm to 300 rpm for cleaning.

20. The method for processing collagen according to claim 18, wherein along a direction of gravity, the first discharge outlet is disposed at a bottom of the compressed air inlet.
